(19) United States
(12) Patent Application Publication
(10) Pub. No.: US 2010/0286762 A1
(43) Pub. Date: Nov. 11, 2010

(54) Compositions and Methods for Ameliorating Clinical Electrical Disturbances

(75) Inventors: Robert G. Gourdie, Charleston, SC (US); Michael P. O'Quinn, Charleston, SC (US); J. Matthew Rhett, Charleston, SC (US)

Correspondence Address: PATENT CORRESPONDENCE;ARNALL GOLDEN GREGORY LLP, 171 17TH STREET NW, SUITE 2100, ATLANTA, GA, 30363, US

(73) Assignee: MUSC Foundation for Research Development, Charleston, SC (US)

(21) Appl. No.: 12/727,000

(22) Filed: Mar. 18, 2010

Related U.S. Application Data
(60) Provisional application No. 61/161,393, filed on Mar. 18, 2009.

(51) Int. Cl. A61F 2/82; A61K 38/17; A61P 9/10; A61P 25/08; A61K 38/22; A61K 9/00; A61K 9/127; A61N 1/02

(52) U.S. Cl. 623/1.42; 514/21.2; 514/16.4; 514/17.7; 514/9.7; 424/484; 424/450; 607/3

(57) ABSTRACT

Disclosed are compositions and methods for the use of ACT1 peptide or other approaches to targeting the ZO-1 PDZ2 domain to treat non-injury related disturbance to electrical activation and ion transients in organ systems.

This application claims the benefit of U.S. Provisional Application No. 61/161,393, filed Mar. 18, 2009, and is herein incorporated by reference in its entirety. This application also incorporates by reference as if rewritten in full the following patent applications: U.S. Provisional Patent Application No. 60/638,366 filed Dec. 21, 2004, U.S. Provisional Application No. 60/671,796 filed Apr. 15, 2005, U.S. Provisional Patent Application No. 60/752,615 filed Dec. 20, 2005, U.S. Utility patent application Ser. No. 11/721,529 filed Dec. 20, 2005, PCT International Patent Application PCT/US2005/46442 filed Dec. 20, 2005, U.S. Utility patent application Ser. No. 11/761,729 filed Jun. 12, 2007, and PCT International Patent Application PCT/US2008/067944 filed Jun. 23, 2008.

### I. BACKGROUND

Previously we have shown that a formulation of ACT1 peptide when applied to the heart coincident with a cryo-infarction to the ventricle renders the heart refractory to arrhythmia-inducing electrophysiological protocols 1 week following the injury (Table 1-O'Quinn et al., Circulation, 2008). Based on these results and a potential mechanism called the “connexon switch” (see model in FIG. 1 below), we put forward the efficacy of ACT1 in suppressing non-injury related arrhythmias. ACT1 peptide or similarly acting compositions can be a part of drug treatment protocols that electrically stabilize the heart reducing death and morbidity rates associated with non-injury related cardiac disease, congenital defect, aging or pathological processes not resulting from injury. ACT peptides are based on linkage of the carboxy-terminal most amino acids of alpha connexin proteins to the cell permeabilization sequence antennapedia. ACT peptides have uses in wound healing and macula degeneration.

### II. SUMMARY

Disclosed are methods and compositions related to electrical disturbances and compositions and methods for modulating them.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a model for ZO-1 PDZ2 regulation of the “Connexon Switch” between Free-Membrane Connexons and Connexons Aggregated in GJs.

FIG. 2 shows ACT2. ACT1-treated Cx43-expressing HeLa cells show decreased hemichannel activity as assayed by live-imaging of ethidium bromide uptake. *=p<0.05 vs wt HeLa §=vs HeLa Cx43.

FIG. 3 shows a Biotinylation assay. HeLa Cx43 cells were treated with ACT1ACT1, REV or VEH control, lysed either immediately following biotinylation (0 hr), or re-incubated at 37° C. for 2 hrs the lysed (2 hr). The top panel is a blot of biotinylated Tx-soluble (upper) and insoluble (lower) Cx43. As expected from normal cycling, PM Cx43 shifts to junctional pools (Tx-100 insoluble) over time (compare 2 hr control to 0 hr). This is enhanced by ACT1, providing biochemical evidence that disruption of Cx43/ZO-1 interaction increases connexon recruitment into GJs.

FIGS. 4A-4F show ZO-1 regulation of differential adhesion between Cx43 expressing cells. A. Segregation of Cx43-GFP cells from DiI-labeled HeLa Cx43 cells. B. ACT1ACT1-mediated reduction in fibroblast (NCRF) adhesion to myocyte (NVRM) monolayers over a 48 hr timecourse. C and D. Cohesion patterns of orange-tagged NVRMs from green-tagged NCRFs following control (top) or ACT1ACT1 (bottom) incubation. E. Fibroblasts are excluded more from ACT1ACT1 incubated aggregates (suggestive of lower cohesion with NVRMs) and F. Cohesion index (% co-localization orange and green) of NVRMs (i.e., NVRMs tend to adhere to each other, rather than NCRFs) is higher following ACT1ACT1 incubation. Scale A=5 um, C, D=25 um.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

Non-injury related disturbance to electrical activation such as atrial fibrillation, irritable bowel syndrome or certain epilepsies are among the most widespread clinical disturbances of activation, causing significant morbidity and mortality. Certain embodiments comprise the use of ACT1 peptide treatment or other approaches to targeting the ZO-1 PDZ2 domain to treat non-injury related disturbance to electrical activation and ion transients in organ systems.

### A. Arrythmia Model

1. A Mechanism Underlying ACT1 Anti-Electrical Disturbance Properties—“the Connexon Switch”

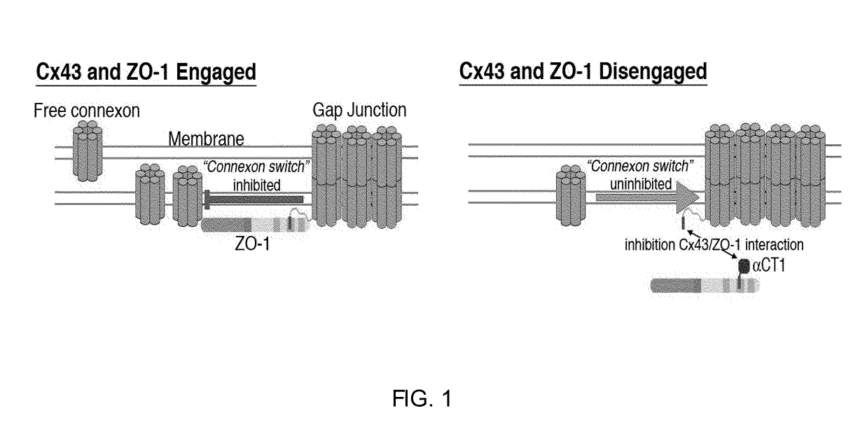

The model in FIG. 1, provides an outline of our premise that ZO-1 PDZ2 regulates the rate of accretion of free connexons in the membrane to gap junctions (GJs)—the “connexon switch”. The model envisages that when ZO-1 engages the Cx43 carboxyl-terminus (CT), recruitment of free connexons into the GJ is inhibited. However, when ZO-1 interaction with Cx43 is disrupted (e.g., by ACT1 or by Cx43-GFP inactivation of the ZO-1 PDZ2 domain) the recruitment of free connexons into the GJ flows uninhibited. Conversely, when Cx43/ZO-1 interaction is increased (e.g., in pathologies such as cardiomyopathy) free connexons may increase and GJs reduce in extent. This new model can account for various previous observations including increases in GJ size and a shift in the proportion of Cx43 in insoluble junctional vs. soluble non-junctional pools (e.g., Hunter et al., Mol Biol Cell, 2005). Moreover, the ZO-1-regulated balance between free non-junctional connexons and GJs is hypothesized to impact arrhythmic potential and/or electrical activation disturbance in various ways including via: (i) coupling dependent-affects on action potential conduction velocity, (ii) Cx43 connexon hemichannel dependent effects on membrane excitability and (iii) cell adhesion dependent effects on heterocellular cohesions (e.g., myocyte-fibroblast interactions) and ECM distribution. This new mechanism explains how ACT1, or other strategies targeting the ZO-1 PDZ2 domain, functions therapeutically in a broad range of cardiac arrhythmias and electrical disturbances of excitable and other tissues (e.g., atrial fibrillation, brain epilepsy and seizure) not related to or caused by injury to those tissues. Data supporting this model is found in the Examples herein.

### B. Methods

Disclosed herein, ACT1 peptide effects membrane excitability and ion transients of the heart, nervous system, uterus and other tissues in health and disease. Disclosed herein, ACT1 acts on connexins, connexons and other channels to modulate excitability and other manifestations of ion transients within cells, between cells and the surrounding extracellular space. This effect can be of use in muscle, nervous and other tissues, where altered ion transients can have consequences such as causing a heart attack or epileptic seizure, irritable bowel syndrome and problematic child birth.

ACT1 or other PDZ2 targeting modalities can be provided orally, intravenously, via implantable biodegradable matrices, such as a with a patch or wafer, or by direct bolus injection into tissues including in conjunction with protease inhibitors, multifunctional polymers, micro-/nanoparticulate drug, polyion complexes, liposomes or other strategies that optimize the release or stability of the drug.

1. Cardiac Arrhythmia

There are many different types of arrhythmia that can lead to abnormal function in the human heart. All forms of arrhythmia have associated morbidity and the potential to result in sudden cardiac death (SCD). Tachyarrhythmias, like ventricular tachycardia and ventricular fibrillation are the predominant mechanisms leading to SCD. In the clinic, SCD is most commonly linked to coronary artery disease and subsequent transient ischemia. These episodes of transient ischemia can induce gap junction remodeling in un-injured tissues, and this remodeling can then cause arrhythmia. Interestingly, only twenty percent of SCD-related autopsies show evidence of a recent, healing, myocardial infarction. In fact, it is more common for these individuals to have had a completely healed infarct prior to the arrhythmogenic incident. There are also several non-ischemic pathologies that can result in SCD. A significant fraction of such arrhythmias may not be associated with myocardial injury per se.

Common arrhythmias include bradycardias, tachycardias, automaticity defects, re-entrant arrhythmias, fibrillation and triggered beats. ACT1 or PDZ2 targeting can be used to treat cardiac rhythm disturbances of these types.

There are many diseases of congenital, genetic and acquired origins that manifest as a primarily electrical pathophysiology. In such cases accompanying tissue injury is not a factor in the generation of the arrhythmogenic substrate. These include, but are not limited to, Long QT syndrome, Short QT syndrome, Brugada syndrome, and several accessory pathway disorders. One example, Wolff-Parkinson-White syndrome (WPW) is a condition where an accessory bundle of muscle, expressing Cx43 gap junctions, links the atrium and the ventricle of the subject. This additional pathway provides the substrate for a reentrant circuit between the atrium and the ventricle which when activated can result in ventricular tachycardia, and potentially lead to SCD. Acute or chronic treatment of the subject with ACT1 peptide will modulate, such as prevent or reduce the likelihood of this reentrant pathway to become activated. This effect can be the result of the peptide's direct modulation of membrane excitability in the region of reentrant activity. Therefore, a dose enabling the delivery of 0.01 to 1000 mg ACT1 peptide per kg body weight to the area of reentrant activity can be used and be beneficial. Administration of the ACT1 compound or an orally available analogue can result in an increase of this compound in the hepatic circulation. Therefore, oral and intravenous administration can provide quick access to the area of reentrant activity for use in acute conditions where the subject might experience and episode of ventricular tachycardia. These administered doses can consist of variants of the peptide to improve stability including amino acid enantomers or related analogues and conservative variants. Chronic administration can also be achieved via these two modalilties or it can be achieved through the implant of a device that slowly releases the ACT1 peptide over a longer period of time.

Arrhythmias can also be the result of molecular abnormalities in the working myocardium. These molecular abnormalities can be caused by the cellular response to environmental stress, genetic mutations, infection, and other conditions. One example of this type of disease is Hypertrophic Cardiomyopathy (HCM). HCM is the number one cause of sudden cardiac death in patients under 30 years of age. This disease can be transmitted genetically and results in the unchecked growth of the myocardium without any signs of injury. It can be diagnosed with a preventative physical exam and/or thorough family history. In this condition, gap junction remodeling in the hypertrophic working myocardium leads to the increased incidence of arrhythmia and can cause SCD. This outcome is often seen as the otherwise healthy young person who suddenly dies after a period of exercise. Examples of such subjects occasionally can be seen in media stories concerning young prominent athletes who die suddenly of an unexpected heart attack. Treatment with ACT1 peptide can prevent the occurrence of unexpected arrhythmias in these subjects. ACT1 peptide can be administered acutely via intracardiac injection of 0.01 to 1000 mg/kg in the case of a subject who suddenly collapses. An increased dose may be necessary to adequately treat the entire myocardium that is affected by this disorder. Chronic administration could be achieved by oral administration of the ACT1 peptide or an orally available analogue at a dose that would enable adequate compound to reach the myocardium. Alternatively, administration can be achieved via an implantable device that slowly releases the ACT1 peptide into the coronary circulation.

In addition, arrhythmias can be the result of reentrant activity or automaticity in the atrium and muscular walls of the large vessels attached to the heart. Atrial fibrillation (AF) is the most common sustained arrhythmia, affecting 5% of Americans, more than 2 million people. In this disease, the subject will present with paroxysmal, transient episodes of fibrillation. Overtime these episodes of uncontrolled atrial rhythm will become more persistent, and there is a possibility that they can even become permanent. Although AF is not deadly on its own, it can lead to several other deadly conditions related to the inherent hypotension and hemostasis it causes. Additionally, atrial action potentials will occasionally leak into the ventricles causing ventricular tachycardia. This can eventually lead to cardiac arrest. It has been suggested that as many as 90% of AF cases are caused by focal activation in the pulmonary veins. Administration of 0.001 to 1000 mg per kg of the ACT1 peptide within the first few minutes of atrial fibrillation could serve to quickly stop the episode and prevent more severe sequelae. For this purpose, oral or intravenous administration of the peptide or its conservatively modified variants would be particularly useful because it would result in quick delivery of the peptide to the affected area. Likewise, continued administration of the peptide via either of these modalities or a device allowing slow release of the peptide can prevent the occurrence of future episodes of atrial fibrillation. For example, ACT1 peptide can be used to coat slow release nanoparticles loaded which can then be injected into the blood stream of the subject. Patients receiving this treatment can be monitored by a Doctor until the arrhythmia resolves and repeated if necessary.

Chronic administration of ACT1 peptide could be useful in preventing the occurrence of all types of arrhythmia. As such, disclosed is a device that slowly releases ACT1 peptide into the myocardium or other regions of the heart. This device can be very helpful for chronic arrhythmogenic conditions. The compositions, such as ACT1 peptide, to be delivered can include: a gel, a methyl cellulose patch, or biodegradable matrix placed against the external surface of the heart, in the pericardial sac, or the pleural space. Alternatively, the peptide can be administered via inhalation into the lungs because of the short circulatory pathway between the lungs and the heart. Other methods of delivery include catheter-based approaches, like implantable stents or expanding devices that would lodge into the coronary circulation or atrial lumen. Several polymer based biodegradable substances have been created that can be optimized to control the concentration and length of peptide administration. All of these devices can be formulated to slowly elute peptide into the heart directly to decrease collateral effects to other parts of the body.

Other common arrhythmias include premature Atrial Contractions, wandering Atrial pacemaker, Multifocal atrial tachycardia, Atrial flutter, Atrial fibrillation, Supraventricular tachycardia, AV nodal reentrant tachycardia is the most common cause of Paroxysmal Supra-ventricular Tachycardia, Junctional rhythm, Junctional tachycardia, Premature junctional complex, Wolff-Parkinson-White syndrome, Lown-Ganong-Levine syndrome, Premature Ventricular Contractions (PVC) sometimes called Ventricular Extra Beats, Accelerated idioventricular rhythm, Monomorphic Ventricular tachycardia, Polymorphic ventricular tachycardia, Ventricular fibrillation, First degree heart block, which manifests as PR prolongation, Second degree heart block, Type 1 Second degree heart block, Type 2 Second degree heart block, Third degree heart block. ACT1 or PDZ2 targeting can be used to treat cardiac rhythm disturbances of these types.

Common drugs used for arrhythmia treatments include class Ia drugs e.g., Quinidine, Procainamide, Disopyramide, class Ib drugs e.g., Lidocaine, Phenyloin, Mexiletine, class Ic drugs e.g., Flecamide, Propafenone, Moricizine, class II drugs e.g., Propranolol, Esmolol, Timolol, Metoprolol and Atenolol, class III drugs e.g., Amiodarone, Sotalol, Ibutilide and Dofetilide, class IV drugs e.g., Verapamil, Diltiazem and class V drugs e.g., Adenosine and Digoxin. ACT1 or PDZ2 targeting can be used in conjunction with these approaches to treatment of arrhythmia.

Other arrhythmia treatments include: Anticoagulant therapies, electrical treatments, electrical cautery, cryo-ablation, radio frequency ablation, implantable cardioverter-defibrillator, and implantable pacemaker. ACT1 or PDZ2 targeting can be used in association with these approaches for the treatment of arrhythmia.

2. Other Non-Injury Related Pathologies of Exciteable Tissues that PDZ2 Targeting would have Therapeutic Use

Epilepsy is a chronic neurological disorder characterized by recurrent, transient, unprovoked seizures, resulting from disturbed neuronal activity in the brain. There is evidence that epilepsy is caused by dysregulated connexin coupling between neuronal cells and disturbances to Cx43 have been noted in human hippocampus associated with severe epilepsy. Over 50 million people worldwide have epilepsy. Over 30% of people with epilepsy do not respond to currently available medications. The uncontrolled electrical disturbance associated with epilepsy often leads to comparisons to cardiac arrhythmias.

Common forms of epilepsy include: Autosomal dominant nocturnal frontal lobe epilepsy, Benign centrotemporal lobe epilepsy of childhood, Benign occipital epilepsy of childhood, Catamenial epilepsy, Childhood absence epilepsy, Dravet's syndrome, Frontal lobe epilepsy, Juvenile absence epilepsy, Juvenile myoclonic epilepsy, Lennox-Gastaut syndrome, Primary reading epilepsy, Progressive myoclonic epilepsies, Rasmussen's encephalitis, Symptomatic localization-related epilepsies, Temporal lobe epilepsy, West syndrome. ACT1 or PDZ2 targeting can be used to treat these epilepsies.

The following medications are used for treatment of epilepsy: carbamazepine, clorazepate (Tranxene) clonazepam (Klonopin), ethosuximide (Zarontin), felbamate (Felbatol), fosphenyloin (Cerebyx), gabapentin (Neurontin), lamotrigine (Lamictal), levetiracetam (Keppra), oxcarbazepine (Trileptal), phenobarbital (Luminal), phenyloin (Dilantin), pregabalin (Lyrica), primidone (Mysoline), tiagabine (Gabitril), topiramate (Topamax), valproate semisodium (Depakote), valproic acid (Depakene), zonisamide (Zonegran), clobazam (Frisium) and vigabatrin (Sabril), retigabine, brivaracetam, and seletracetam, diazepam (Valium, Diastat) and lorazepam (Ativan), Paral, midazolam (Versed), and pentobarbital (Nembutal), acetazolamide (Diamox), progesterone, adrenocorticotropic hormone (ACTH, Acthar), various corticotropic steroid hormones (prednisone), or bromide. ACT1 or PDZ2 targeting can be used in association with these drugs in treatment of epilepsy.

Other epilepsy treatments include: ketogenic diet, electrical stimulation, vagus nerve stimulation, responsive neurostimulator system (rns), deep brain stimulation, invasive or noninvasive surgery, avoidance therapy, warning systems, alternative or complementary medicine. ACT1 or PDZ2 targeting can be used in association with these approaches to treatment of epilepsy.

Irritable Bowel Syndrome, chronic intestinal pseudo-obstruction and other diseases of altered intestinal motility can also be treated with the ACT1 peptide or other compositions that inhibit the CX43 interaction at the PDZ2 domain. See Doring, B et al Cell tissue res. 2007 (327)333-342.

Finally, Cx43 expression is upregulated in the mammalian uterus during pregnancy, and is necessary for embryo implantation. In addition increased levels of Cx43 are necessary for uterine contraction at the time of child birth. ACT1 peptide or similarly acting compositions can promote stronger uterine contractions and lead to a reduction in cesarean sections and uterine tears at the time of birth

### C. Effective Dosages

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual doctor in the event of any counter indications. Dosage can vary, and can be administered in one or more dose administrations daily, for one or several days. Guidance can be found in the literature for appropriate dosages for given classes of pharmaceutical products. The range of dosage largely depends on the application of the compositions herein, severity of condition, and its route of administration. For example, in applications as a laboratory tool for research, the ACT peptide compositions can be used in doses as low as 0.01% w/v. The dosage can be as low as 0.02% w/v and possibly as high as 2% w/v in topical treatments. Significantly higher concentrations of the compositions by themselves or in combination with other compounds may be used in applications like early concentrated bolus immediately during an acute epileptic seizure. Thus, upper limits of the provided polypeptides may be up to 1000 mg/kg if given as an initial bolus delivered for example directly into the heart. Recommended upper limits of dosage for parenteral routes of administration for example intramuscular, intracerebral, intracardicardiac and intraspinal could be up to 500 mg/kg depending on the severity of the electrical disturbance. This lower dosage limit may vary by formulation, depending for example on how the polypeptide(s) is combined with other agents promoting its action or acting in concert with the polypeptide(s). For continuous delivery of the provided polypeptides, for example, in combination with an intravenous drip, lower limits of 0.01 g/Kg body weight over time courses determined by the doctor based on improvement in the condition can be used. In another example, upper limits of concentration of nucleic acids provided to target ZO-1 PDZ2 delivered internally for example, intramuscular, intracerebral, intracardicardiac and intraspinal would be 50-100 μg/ml of solution. Again, the frequency would be determined by the Doctor based on improvement.

1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### D. Specific Embodiments

Disclosed are methods of treating a subject for membrane excitability comprising administering a PDZ2 targeting modality.

Also disclosed are methods, wherein the PDZ2 targeting modality comprises a conservatively modified variant, amino acid enantiomer or analogue of ACT1 peptide, wherein the conservatively modified variant of ACT1 peptide comprises the ACT1 peptide, wherein the membrane excitability is of the heart, nervous system, muscle, uterus, wherein the subject is being treated for heart attack, epileptic seizure, irritable bowel syndrome, or problematic child birth or alone or in any combination with any methods or compositions disclosed herein.

Disclosed are methods, wherein the PDZ2 targeting modality is delivered orally, intravenously, with an implantable biodegradable matrice, with a gel, with a patch, with a methyl cellulose patch with a wafer, by direct bolus injection into tissues, through a multifunctional polymer, through a micro-/nanoparticulate drug, through a polyion complex, through a liposome, in conjunction with protease inhibitors, a slow release implantable device, catheter-based approaches, through an implantable stent, or through an expanding device or alone or in any combination with any methods or compositions disclosed herein.

Also disclosed are methods, wherein the membrane excitability is associated with a tissue arrhythmia, wherein the tissue arrhythmia is a cardiac arrhythmia, wherein the cardiac arrhythmia is ventricular tachycardia, ventricular fibrillation, atrial fibrillation, bradycardia, tachycardia, automaticity defect, re-entrant arrhythmia, fibrillation, or triggered beats, premature Atrial Contractions, wandering Atrial pacemaker, Multifocal atrial tachycardia, Atrial flutter, Atrial fibrillation, Supraventricular tachycardia, AV nodal reentrant tachycardia is the most common cause of Paroxysmal Supra-ventricular Tachycardia, Junctional rhythm, Junctional tachycardia, Premature junctional complex, Wolff-Parkinson-White syndrome, Lown-Ganong-Levine syndrome, Premature Ventricular Contractions (PVC) sometimes called Ventricular Extra Beats, Accelerated idioventricular rhythm, Monomorphic Ventricular tachycardia, Polymorphic ventricular tachycardia, Ventricular fibrillation, First degree heart block, which manifests as PR prolongation, Second degree heart block, Type 1 Second degree heart block, Type 2 Second degree heart block, or Third degree heart block, wherein the membrane excitability is associated with an electrical pathophysiology, wherein electrical pathophysiolgy is a Long QT syndrome, Short QT syndrome, Brugada syndrome, several accessory pathway disorder, Wolff-Parkinson-White syndrome (WPW), Hypertrophic Cardiomyopathy, epilepsy, Autosomal dominant nocturnal frontal lobe epilepsy, Benign centrotemporal lobe epilepsy of childhood, Benign occipital epilepsy of childhood, Catamenial epilepsy, Childhood absence epilepsy, Dravet's syndrome, Frontal lobe epilepsy, Juvenile absence epilepsy, Juvenile myoclonic epilepsy, Lennox-Gastaut syndrome, Primary reading epilepsy, Progressive myoclonic epilepsy, Rasmussen's encephalitis, Symptomatic localization-related epilepsies, Temporal lobe epilepsy, or West syndrome, or alone or in any combination with any methods or compositions disclosed herein.

Also disclosed are methods, wherein the PDZ2 targeting modality comprises a formulation that delivers 0.001 to 1000 mg per kg body weight to the area of membrane excitability or a reentrant activity, wherein the delivery occurs by being placed against the external surface of the heart, in the pericardial sac, the pleural space or through inhalation into the lungs, or alone or in any combination with any methods or compositions disclosed herein.

Also disclosed are methods, further comprises administering a second arrhythmia treatment, wherein the second arrhythmia treatment comprises administering Quinidine, Procainamide, Disopyramide, class Ib drug, Lidocaine, Phenyloin, Mexiletine, class Ic drug, Flecamide, Propafenone, Moricizine, class II drug, Propranolol, Esmolol, Timolol, Metoprolol and Atenolol, class III drug, Amiodarone, Sotalol, Ibutilide and Dofetilide, class IV drug, Verapamil, Diltiazem and class V drug, Adenosine, or Digoxin, performing an Anticoagulant therapy, electrical treatment, electrical cautery, cryo-ablation, radio frequency ablation, implantable cardioverter-defibrillator, or implantable pacemaker, wherein the second arrhythmia treatment comprises carbamazepine, clorazepate (Tranxene) clonazepam (Klonopin), ethosuximide (Zarontin), felbamate (Felbatol), fosphenyloin (Cerebyx), gabapentin (Neurontin), lamotrigine (Lamictal), levetiracetam (Keppra), oxcarbazepine (Trileptal), phenobarbital (Luminal), phenyloin (Dilantin), pregabalin (Lyrica), primidone (Mysoline), tiagabine (Gabitril), topiramate (Topamax), valproate semisodium (Depakote), valproic acid (Depakene), zonisamide (Zonegran), clobazam (Frisium) and vigabatrin (Sabril), retigabine, brivaracetam, and seletracetam, diazepam (Valium, Diastat) and lorazepam (Ativan), Paral, midazolam (Versed), and pentobarbital (Nembutal), acetazolamide (Diamox), progesterone, adrenocorticotropic hormone (ACTH, Acthar), various corticotropic steroid hormones (prednisone), bromide, ketogenic diet, electrical stimulation, vagus nerve stimulation, responsive neurostimulator system (rns), deep brain stimulation, invasive or noninvasive surgery, avoidance therapy, warning systems, alternative or complementary medicine, or alone or in any combination with any methods or compositions disclosed herein.

Also disclosed are formulations of a PDZ2 targeting modality comprising a patch for directly delivery to a heart.

Also disclosed are devices comprising a long term release mechanism for delivery of a PDZ2 targeting modality.

Also disclosed are methods of producing the PDZ2 targeting modality of any of the methods or compositions disclosed herein.

### E. Compositions

There are a variety compositions and traits and characteristics related to compositions which are discussed herein.

1. Homology/Identity

It is understood that one way to define any known variants and derivatives or those that might arise, of the disclosed genes and proteins herein is through defining the variants and derivatives in terms of homology to specific known sequences. For example SEQ ID NO:1 sets forth a particular sequence of an ACT1 protein. Those of skill in the art readily understand how to determine the homology of two proteins or nucleic acids, such as genes. For example, the homology can be calculated after aligning the two sequences so that the homology is at its highest level.

2. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

3. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, in some embodiments selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, in some embodiments selective hybridization conditions would be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Preferred conditions also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

4. Nucleic Acids

There are a variety of molecules disclosed herein that are nucleic acid based, including for example the nucleic acids that encode, for example, ACT1 as well as any other proteins disclosed herein, as well as various functional nucleic acids. The disclosed nucleic acids are made up of for example, nucleotides, nucleotide analogs, or nucleotide substitutes. Non-limiting examples of these and other molecules are discussed herein. It is understood that for example, when a vector is expressed in a cell, that the expressed mRNA will typically be made up of A, C, G, and U. Likewise, it is understood that if, for example, an antisense molecule is introduced into a cell or cell environment through for example exogenous delivery, it is advantageous that the antisense molecule be made up of nucleotide analogs that reduce the degradation of the antisense molecule in the cellular environment.

a) Nucleotides and Related Molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate).

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556).

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

b) Sequences

There are a variety of sequences related to, for example, ACT1 as well as any other protein disclosed herein that are disclosed on Genbank, and these sequences and others are herein incorporated by reference in their entireties as well as for individual subsequences contained therein.

A variety of sequences are provided herein and these and others can be found in Genbank, at www.pubmed.gov. Those of skill in the art understand how to resolve sequence discrepancies and differences and to adjust the compositions and methods relating to a particular sequence to other related sequences. Primers and/or probes can be designed for any sequence given the information disclosed herein and known in the art.

c) Primers and Probes

Disclosed are compositions including primers and probes, which are capable of interacting with the genes disclosed herein. In certain embodiments the primers are used to support DNA amplification reactions. Typically the primers will be capable of being extended in a sequence specific manner. Extension of a primer in a sequence specific manner includes any methods wherein the sequence and/or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the nucleic acid or region of the nucleic acid or they hybridize with the complement of the nucleic acid or complement of a region of the nucleic acid.

d) Functional Nucleic Acids

Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of a PDZ2 domain or the genomic DNA of a PDZ2 domain or they can interact with the polypeptide encoding a PDZ2 domain. These can be called PDZ2 targeting modalities. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Antisense molecules are designed to interact with a target nucleic acid molecule through either canonical or non-canonical base pairing. The interaction of the antisense molecule and the target molecule is designed to promote the destruction of the target molecule through, for example, RNAseH mediated RNA-DNA hybrid degradation. Alternatively the antisense molecule is designed to interrupt a processing function that normally would take place on the target molecule, such as transcription or replication. Antisense molecules can be designed based on the sequence of the target molecule. Numerous methods for optimization of antisense efficiency by finding the most accessible regions of the target molecule exist. Exemplary methods would be in vitro selection experiments and DNA modification studies using DMS and DEPC. It is preferred that antisense molecules bind the target molecule with a dissociation constant (k_{d}) less than or equal to 10⁻⁶, 10⁻⁸, 10⁻¹⁰, or 10⁻¹². A representative sample of methods and techniques which aid in the design and use of antisense molecules can be found in the following non-limiting list of U.S. Pat. Nos. 5,135,917, 5,294,533, 5,627,158, 5,641,754, 5,691,317, 5,780,607, 5,786,138, 5,849,903, 5,856,103, 5,919,772, 5,955,590, 5,990,088, 5,994,320, 5,998,602, 6,005,095, 6,007,995, 6,013,522, 6,017,898, 6,018,042, 6,025,198, 6,033,910, 6,040,296, 6,046,004, 6,046,319, and 6,057,437.

Aptamers are molecules that interact with a target molecule, preferably in a specific way. Typically aptamers are small nucleic acids ranging from 15-50 bases in length that fold into defined secondary and tertiary structures, such as stem-loops or G-quartets. Aptamers can bind small molecules, such as ATP (U.S. Pat. No. 5,631,146) and theophiline (U.S. Pat. No. 5,580,737), as well as large molecules, such as reverse transcriptase (U.S. Pat. No. 5,786,462) and thrombin (U.S. Pat. No. 5,543,293). Aptamers can bind very tightly with k_{d}s from the target molecule of less than 10⁻¹²M. It is preferred that the aptamers bind the target molecule with a k_{d} less than 10⁻⁶, 10⁻⁸, 10⁻¹⁰, or 10⁻¹². Aptamers can bind the target molecule with a very high degree of specificity. For example, aptamers have been isolated that have greater than a 10000 fold difference in binding affinities between the target molecule and another molecule that differ at only a single position on the molecule (U.S. Pat. No. 5,543,293). It is preferred that the aptamer have a k_{d} with the target molecule at least 10, 100, 1000, 10,000, or 100,000 fold lower than the k_{d} with a background binding molecule. It is preferred when doing the comparison for a polypeptide for example, that the background molecule be a different polypeptide. For example, when determining the specificity of PDZ2 domain aptamers, the background protein could be serum albumin Representative examples of how to make and use aptamers to bind a variety of different target molecules can be found in the following non-limiting list of U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698.

Ribozymes are nucleic acid molecules that are capable of catalyzing a chemical reaction, either intramolecularly or intermolecularly. Ribozymes are thus catalytic nucleic acid. It is preferred that the ribozymes catalyze intermolecular reactions. There are a number of different types of ribozymes that catalyze nuclease or nucleic acid polymerase type reactions which are based on ribozymes found in natural systems, such as hammerhead ribozymes, (for example, but not limited to the following U.S. Pat. Nos. 5,334,711, 5,436,330, 5,616,466, 5,633,133, 5,646,020, 5,652,094, 5,712,384, 5,770,715, 5,856,463, 5,861,288, 5,891,683, 5,891,684, 5,985,621, 5,989,908, 5,998,193, 5,998,203, WO 9858058 by Ludwig and Sproat, WO 9858057 by Ludwig and Sproat, and WO 9718312 by Ludwig and Sproat) hairpin ribozymes (for example, but not limited to the following U.S. Pat. Nos. 5,631,115, 5,646,031, 5,683,902, 5,712,384, 5,856,188, 5,866,701, 5,869,339, and 6,022,962), and tetrahymena ribozymes (for example, but not limited to the following U.S. Pat. Nos. 5,595,873 and 5,652,107). There are also a number of ribozymes that are not found in natural systems, but which have been engineered to catalyze specific reactions de novo (for example, but not limited to the following U.S. Pat. Nos. 5,580,967, 5,688,670, 5,807,718, and 5,910,408). Preferred ribozymes cleave RNA or DNA substrates, and more preferably cleave RNA substrates. Ribozymes typically cleave nucleic acid substrates through recognition and binding of the target substrate with subsequent cleavage. This recognition is often based mostly on canonical or non-canonical base pair interactions. This property makes ribozymes particularly good candidates for target specific cleavage of nucleic acids because recognition of the target substrate is based on the target substrates sequence. Representative examples of how to make and use ribozymes to catalyze a variety of different reactions can be found in the following non-limiting list of U.S. Pat. Nos. 5,646,042, 5,693,535, 5,731,295, 5,811,300, 5,837,855, 5,869,253, 5,877,021, 5,877,022, 5,972,699, 5,972,704, 5,989,906, and 6,017,756.

Triplex forming functional nucleic acid molecules are molecules that can interact with either double-stranded or single-stranded nucleic acid. When triplex molecules interact with a target region, a structure called a triplex is formed, in which there are three strands of DNA forming a complex dependant on both Watson-Crick and Hoogsteen base-pairing. Triplex molecules are preferred because they can bind target regions with high affinity and specificity. It is preferred that the triplex forming molecules bind the target molecule with a k_{d} less than 10⁻⁶, 10⁻⁸, 10⁻¹⁰, or 10⁻¹². Representative examples of how to make and use triplex forming molecules to bind a variety of different target molecules can be found in the following non-limiting list of U.S. Pat. Nos. 5,176,996, 5,645,985, 5,650,316, 5,683,874, 5,693,773, 5,834,185, 5,869,246, 5,874,566, and 5,962,426.

External guide sequences (EGSs) are molecules that bind a target nucleic acid molecule forming a complex, and this complex is recognized by RNase P, which cleaves the target molecule. EGSs can be designed to specifically target a RNA molecule of choice. RNAse P aids in processing transfer RNA (tRNA) within a cell. Bacterial RNAse P can be recruited to cleave virtually any RNA sequence by using an EGS that causes the target RNA:EGS complex to mimic the natural tRNA substrate. (WO 92/03566 by Yale, and Forster and Altman, Science 238:407-409 (1990)).

Similarly, eukaryotic EGS/RNAse P-directed cleavage of RNA can be utilized to cleave desired targets within eukaryotic cells. (Yuan et al., Proc. Natl. Acad. Sci. USA 89:8006-8010 (1992); WO 93/22434 by Yale; WO 95/24489 by Yale; Yuan and Altman, EMBO J 14:159-168 (1995), and Carrara et al., Proc. Natl. Acad. Sci. (USA) 92:2627-2631 (1995)). Representative examples of how to make and use EGS molecules to facilitate cleavage of a variety of different target molecules can be found in the following non-limiting list of U.S. Pat. Nos. 5,168,053, 5,624,824, 5,683,873, 5,728,521, 5,869,248, and 5,877,162.

5. Nucleic Acid Delivery

In the methods described above which include the administration and uptake of exogenous DNA into the cells of a subject (i.e., gene transduction or transfection), the disclosed nucleic acids can be in the form of naked DNA or RNA, or the nucleic acids can be in a vector for delivering the nucleic acids to the cells, whereby the antibody-encoding DNA fragment is under the transcriptional regulation of a promoter, as would be well understood by one of ordinary skill in the art. The vector can be a commercially available preparation, such as an adenovirus vector (Quantum Biotechnologies, Inc. (Laval, Quebec, Canada). Delivery of the nucleic acid or vector to cells can be via a variety of mechanisms. As one example, delivery can be via a liposome, using commercially available liposome preparations such as LIPOFECTIN, LIPOFECTAMINE (GIBCO-BRL, Inc., Gaithersburg, Md.), SUPERFECT (Qiagen, Inc. Hilden, Germany) and TRANSFECTAM (Promega Biotec, Inc., Madison, Wis.), as well as other liposomes developed according to procedures standard in the art. In addition, the disclosed nucleic acid or vector can be delivered in vivo by electroporation, the technology for which is available from Genetronics, Inc. (San Diego, Calif.) as well as by means of a SONOPORATION machine (ImaRxPharmaceutical Corp., Tucson, Ariz.).

As one example, vector delivery can be via a viral system, such as a retroviral vector system which can package a recombinant retroviral genome (see e.g., Pastan et al., Proc. Natl. Acad. Sci. U.S.A. 85:4486, 1988; Miller et al., Mol. Cell. Biol. 6:2895, 1986). The recombinant retrovirus can then be used to infect and thereby deliver to the infected cells nucleic acid encoding a broadly neutralizing antibody (or active fragment thereof). The exact method of introducing the altered nucleic acid into mammalian cells is, of course, not limited to the use of retroviral vectors. Other techniques are widely available for this procedure including the use of adenoviral vectors (Mitani et al., Hum. Gene Ther. 5:941-948, 1994), adeno-associated viral (AAV) vectors (Goodman et al., Blood 84:1492-1500, 1994), lentiviral vectors (Naldini et al., Science 272:263-267, 1996), pseudotyped retroviral vectors (Agrawal et al., Exper. Hematol. 24:738-747, 1996). Physical transduction techniques can also be used, such as liposome delivery and receptor-mediated and other endocytosis mechanisms (see, for example, Schwartzenberger et al., Blood 87:472-478, 1996). This disclosed compositions and methods can be used in conjunction with any of these or other commonly used gene transfer methods.

As one example, if the antibody-encoding nucleic acid is delivered to the cells of a subject in an adenovirus vector, the dosage for administration of adenovirus to humans can range from about 10⁷ to 10⁹ plaque forming units (pfu) per injection but can be as high as 10¹² pfu per injection (Crystal, Hum. Gene Ther. 8:985-1001, 1997; Alvarez and Curiel, Hum. Gene Ther. 8:597-613, 1997). A subject can receive a single injection, or, if additional injections are necessary, they can be repeated at six month intervals (or other appropriate time intervals, as determined by the skilled practitioner) for an indefinite period and/or until the efficacy of the treatment has been established.

Parenteral administration of the nucleic acid or vector, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein. For additional discussion of suitable formulations and various routes of administration of therapeutic compounds, see, e.g., Remington: The Science and Practice of Pharmacy (19th ed.) ed. A. R. Gennaro, Mack Publishing Company, Easton, Pa. 1995.

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

6. Peptides

a) Protein Variants

As discussed herein there are numerous variants of the ACT1 protein that are known and herein contemplated. In addition, to the known functional ACT1 strain variants there are derivatives of the ACT1 proteins which also function in the disclosed methods and compositions. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| Amino Acid | Abbreviations |
|---|---|
| alanine | AlaA |
| allosoleucine | AIle |
| arginine | ArgR |
| asparagine | AsnN |
| aspartic acid | AspD |
| cysteine | CysC |
| glutamic acid | GluE |
| glutamine | GlnK |
| glycine | GlyG |
| histidine | HisH |
| isolelucine | IleI |
| leucine | LeuL |
| lysine | LysK |
| phenylalanine | PheF |
| proline | ProP |
| pyroglutamic acidp | Glu |
| serine | SerS |
| threonine | ThrT |
| tyrosine | TyrY |
| tryptophan | TrpW |
| valine | ValV |

TABLE 2
Amino Acid Substitutions

| | Exemplary Conservative Substitutions, |
|---|---|
| Original Residue | others are known in the art. |
| Ala | ser |
| Arg | lys, gln |
| Asn | gln; his |
| Asp | glu |
| Cys | ser |
| Gln | asn, lys |
| Glu | asp |
| Gly | pro |
| His | asn; gln |
| Ile | leu; val |
| Leu | ile; val |
| Lys | arg; gln; |
| Met | Leu; ile |
| Phe | met; leu; tyr |
| Ser | thr |
| Thr | ser |
| Trp | tyr |
| Tyr | trp; phe |
| Val | ile; leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W.H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CHH₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appin, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann Rev. Biochem. 61:387 (1992), incorporated herein by reference).

7. Antibodies

(1) Antibodies Generally

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with a PDZ2 domain such that electrical disturbances in tissues are inhibited. Antibodies that bind the disclosed regions of a PDZ2 domain involved in the interaction with connexxons are also disclosed. The antibodies can be tested for their desired activity using the in vitro assays described herein, or by analogous methods, after which their in vivo therapeutic and/or prophylactic activities are tested according to known clinical testing methods.

The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces mono clonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro, e.g., using the HIV Env-CD4-co-receptor complexes described herein.

The monoclonal antibodies may also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response. The disclosed human antibodies can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies (and fragments thereof) can also be produced using phage display libraries (Hoogenboom et al., J. Mol. Biol., 227:381, 1991; Marks et al., J. Mol. Biol., 222:581, 1991).

(2) Administration of Antibodies

Administration of the antibodies can be done as disclosed herein. Nucleic acid approaches for antibody delivery also exist. The broadly neutralizing anti PDZ2 domain antibodies and antibody fragments can also be administered to patients or subjects as a nucleic acid preparation (e.g., DNA or RNA) that encodes the antibody or antibody fragment, such that the patient's or subject's own cells take up the nucleic acid and produce and secrete the encoded antibody or antibody fragment. The delivery of the nucleic acid can be by any means, as disclosed herein, for example.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389.

Following administration of a disclosed composition, such as an antibody, for treating, inhibiting, or preventing an electrical disturbance, the efficacy of the therapeutic antibody can be assessed in various ways well known to the skilled practitioner. For instance, one of ordinary skill in the art will understand that a composition, such as an antibody, disclosed herein is efficacious in treating or inhibiting an electrical disturbance in a subject by observing that the composition reduces viral load or prevents a further increase in, for example, a fibrillation or arrhythmia.

The compositions that inhibit electrical disturbances disclosed herein may be administered prophylactically to patients or subjects who are at risk for such.

8. Screening

Screening molecules similar to ACT1 for inhibition of an electrical disturbance is a method of isolating desired compounds.

Molecules isolated which inhibit an electrical disturbance can either be competitive inhibitors or non-competitive inhibitors of a PDZ2 domain.

As used herein combinatorial methods and libraries included traditional screening methods and libraries as well as methods and libraries used in interative processes. The assays and tests disclosed herein can be used to test unknown compounds and these methods can include a step of comparing the activity of the unknown compound to the activity of ACT1 in the same assay.

Although described above with reference to design and generation of compounds which could alter binding, one could also screen libraries of known compounds, including natural products or synthetic chemicals, and biologically active materials, including proteins, for compounds which alter PDZ2 domain binding or electrical disturbances.

### F. Methods of Making the Compositions

The compositions disclosed herein and the compositions necessary to perform the disclosed methods can be made using any method known to those of skill in the art for that particular reagent or compound unless otherwise specifically noted.

1. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

2. Peptide Synthesis

One method of producing the disclosed proteins, such as SEQ ID NO:23, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant G A (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

3. Process Claims for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

### G. Definitions

1. A, an, the

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

2. About

About modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

3. ACT1

ACT1 can have the sequence SEQ ID NO:1 RPRPDDLEI. The ACT1 can be the CT sequence from Cx43. The ACT1 sequence can be attached to a cell penetration sequence, such as the antennapedia sequence, such as SEQ ID NO:2 RQPKIWFPNRRKPWKK. A combined PS+ACT1 is SEQ ID NO3: RQPKIWFPNRRKPWKK RPRPDDLEI.

4. Components

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular ACT1 is disclosed and discussed and a number of modifications that can be made to a number of molecules including the ACT1 are discussed, specifically contemplated is each and every combination and permutation of ACT1 and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

5. Comprise

Throughout the description and claims of this specification, the word “comprise” and variations of the word, such as “comprising” and “comprises,” means “including but not limited to,” and is not intended to exclude, for example, other additives, components, integers or steps.

6. Contacting

Contacting or like terms means bringing into proximity such that a molecular interaction can take place, if a molecular interaction is possible between at least two things, such as molecules, cells, markers, at least a compound or composition, or at least two compositions, or any of these with an article(s) or with a machine. For example, contacting refers to bringing at least two compositions, molecules, articles, or things into contact, i.e. such that they are in proximity to mix or touch. For example, having a patch with ACT1 A and a heart tissue then, for example, placing the patch onto the heart tissue would be bringing the patch into contact with the heart tissue. Contacting a cell with a ligand would be bringing a ligand to the cell to ensure the cell have access to the ligand.

It is understood that anything disclosed herein can be brought into contact with anything else.

7. Control

The terms “control” or “control levels” or “control cells” are defined as the standard by which a change is measured, for example, the controls are not subjected to the experiment, but are instead subjected to a defined set of parameters, or the controls are based on pre- or post-treatment levels. They can either be run in parallel with or before or after a test run, or they can be a pre-determined standard. Any of the assays or methods herein can be run with a control.

8. Higher

The terms “higher,” “increases,” “elevates,” or “elevation” or variants of these terms, refer to increases above basal levels, e.g., as compared to a control. The terms “low,” “lower,” “reduces,” or “reduction” or variation of these terms, refer to decreases below basal levels, e.g., as compared to a control. For example, basal levels are normal in vivo levels prior to, or in the absence of, or addition of an agent such as an agonist or antagonist to activity. Any of the assays and methods disclosed herein can be comparative having using the terms, such as increases.

9. Inhibit

By “inhibit” or other forms of inhibit means to hinder or restrain a particular characteristic. It is understood that this is typically in relation to some standard or expected value, in other words it is relative, but that it is not always necessary for the standard or relative value to be referred to. For example, “inhibits electrical disturbance” means hindering or restraining the amount of electrical disturbance that takes place relative to a standard or a control. Any of the assays and methods disclosed herein can be comparative having using the terms, such as inhibit.

10. Material

Material is the tangible part of something (chemical, biochemical, biological, or mixed) that goes into the makeup of a physical object.

11. Molecule

As used herein, the terms “molecule” or like terms refers to a biological or biochemical or chemical entity that exists in the form of a chemical molecule or molecule with a definite molecular weight. A molecule or like terms is a chemical, biochemical or biological molecule, regardless of its size.

Many molecules are of the type referred to as organic molecules (molecules containing carbon atoms, among others, connected by covalent bonds), although some molecules do not contain carbon (including simple molecular gases such as molecular oxygen and more complex molecules such as some sulfur-based polymers). The general term “molecule” includes numerous descriptive classes or groups of molecules, such as proteins, nucleic acids, carbohydrates, steroids, organic pharmaceuticals, small molecule, receptors, antibodies, and lipids. When appropriate, one or more of these more descriptive terms (many of which, such as “protein,” themselves describe overlapping groups of molecules) will be used herein because of application of the method to a subgroup of molecules, without detracting from the intent to have such molecules be representative of both the general class “molecules” and the named subclass, such as proteins. Unless specifically indicated, the word “molecule” would include the specific molecule and salts thereof, such as pharmaceutically acceptable salts.

12. Optionally

“Optional” or “optionally” means that the subsequently described event or circumstance may or may not occur, and that the description includes instances where said event or circumstance occurs and instances where it does not.

13. Prevent

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibits electrical disturbance is disclosed, then reduces and prevents electrical disturbance are also disclosed. Any of the assays and methods disclosed herein can be comparative having using the terms, such as prevent.

14. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data are provided in a number of different formats, and that this data, represents endpoints and starting points, and ranges for any combination of the data points. For example, if a particular datum point “10” and a particular datum point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

15. Reduce

By “reduce” or other forms of reduce means lowering of an event or characteristic. It is understood that this is typically in relation to some standard or expected value, in other words it is relative, but that it is not always necessary for the standard or relative value to be referred to. For example, “reduces phosphorylation” means lowering the amount of phosphorylation that takes place relative to a standard or a control. Any of the assays and methods disclosed herein can be comparative having using the terms, such as reduce.

16. References

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

17. Subject

As used throughout, by a “subject” is meant an individual. Thus, the “subject” can include, for example, domesticated animals, such as cats, dogs, etc., livestock (e.g., cattle, horses, pigs, sheep, goats, etc.), laboratory animals (e.g., mouse, rabbit, rat, guinea pig, etc.) mammals, non-human mammals, primates, non-human primates, rodents, birds, reptiles, amphibians, fish, and any other animal. The subject can be a mammal such as a primate or a human. The subject can also be a non-human.

18. Substance

A substance or like terms is any physical object. A material is a substance. Molecules, ligands, markers, cells, proteins, and DNA can be considered substances. A machine or an article would be considered to be made of substances, rather than considered a substance themselves.

19. Tissue

Tissue or like terms refers to a collection of cells. Typically a tissue is obtained from a subject or manipulated in a subject.

20. Treating

“Treating” or “treatment” does not mean a complete cure. It means that the symptoms of the underlying disease are reduced, and/or that one or more of the underlying cellular, physiological, or biochemical causes or mechanisms causing the symptoms are reduced. It is understood that reduced, as used in this context, means relative to the state of the disease, including the molecular state of the disease, not just the physiological state of the disease. In certain situations a treatment can inadvertantly cause harm.

21. Values

Specific and preferred values disclosed for components, ingredients, additives, cell types, markers, and like aspects, and ranges thereof, are for illustration only; they do not exclude other defined values or other values within defined ranges. The compositions, apparatus, and methods of the disclosure include those having any value or any combination of the values, specific values, more specific values, and preferred values described herein.

Thus, the disclosed methods, compositions, articles, and machines, can be combined in a manner to comprise, consist of, or consist essentially of, the various components, steps, molecules, and composition, and the like, discussed herein. They can be used, for example, in methods for characterizing a molecule including a ligand as defined herein; a method of producing an index as defined herein; or a method of drug discovery as defined herein.

### H. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

Table 1 ACT1 peptide inhibits arrhythmic propensity in cryoinjured mouse hearts.

Following cryoinjury by application of 3 mm liquid nitrogen chilled probe hearts in anesthetized mice were treated with a methycellulose patch containing either ˜9 ug of ACT1 peptide, ˜9 ug of reverse control peptide or no peptide (vehicle control). Mice were then allowed to recover for a week. A week following the injury, Langherdorf-perfused hearts from the mice were subject to 2 types of electrical stimulation inducing arrhythmia: a) S2-S3 premature beat protocol and b) Overdrive pacing. Electrical recordings were made and induced arrhythmic behaviors were categorized including delayed after depolarizations, ectopic beats, ventricular tachycardia, and ventricular fibrillation. The table shows the numbers of mice in each treatment and control groups displaying an arrhythmia response to a given protocol, as well as mean severity levels of arrhythmia within the groups. Severity was graded according to the electrical abnormality noted (e.g. ventricular tachycardia was scored lower than ventricular fibrillation) and the persistence of the electrical abnormality (e.g. intermittent tachycardia was scored lower than persistent tachycardia). Note that at a mean score of 1.71 (yellow highlight), the severity of arrhythmias in the ACT1 treated group is highly significantly (t-test p=0.01 or below) lower than that of either the reverse control or vehicle control groups. Note also that ACT1 peptide treated hearts showed significantly (chi squares p=0.05 or below) improved electrical stability in response to the S2-S3 arrhythmia induction protocol. A similar trend is seen in response to overdrive pacing, although in this case the difference between the treatment and controls are not significant at p=0.05. The “All control” group represents pooled reverse and vehicle control counts.

| | S2-S3 Protocol | | Overdrive Pacing | | Arrhythmia Severity | |
|---|---|---|---|---|---|---|
| Index | Arrhythmic | Stable | Arrhythmia | Stable | Mean | st dev |
| ACT | 1 | 6 | 2 | 5 | 1.71 | 2.56 |
| Vehicle Cont | 5 | 2* | 5 | 2 ns | 5.71** | 3.73 |
| Rev Cont | 6 | 2^{#} | 5 | 3 ns | 5.00^{##} | 3.21 |
| All Control | 11 | 4^{&} | 10 | 5 ns | 5.36^{&&} | 3.75 |

*^{,#,&}= ACT1 vs Control p < 0.05;
**^{,##,&&}= ACT1 vs Control p < 0.01.

### 2. Example 2

### Data Supporting the ZO-1 PDZ2 Mediated “Connexon Switch”

a) ACT1 Increases GJ Intercellular Communication, Reduces Cx43 Hemichannel Activity and Surface Biotin-Labeled Cx43 Connexons:

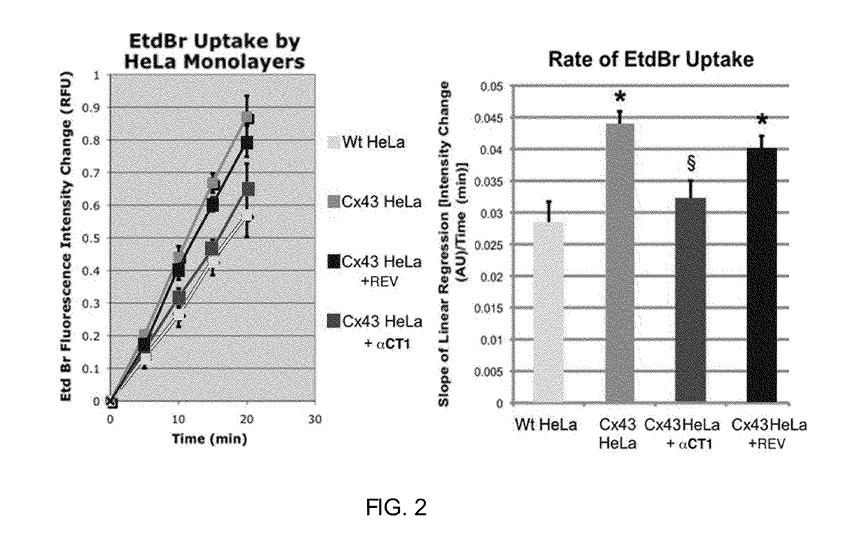

It has been previously shown that inhibition of ZO-1 interaction with Cx43 increases GJ size in HeLa Cx43 and increases the proportion of Triton-insoluble-Cx43 in GJs, without altering Cx43 expression levels or turnover (Hunter et al., 2005). Based on this and other data, it was proposed that ZO-1 inhibits the transition from non-junctional and gap junctional Cx43 (Hunter et al. 2005). The question of where ZO-1 exerted this control to this point is unknown. Here, it is disclosed that ZO-1 regulates movement of connexons in the plasma membrane into GJs. FIG. 1 outlines how this works. The model disclosed herein predicts that inhibiting Cx43/ZO-1 interaction will result in an increase in GJ intercellular communication (GJIC) and a complementary decrease of free-membrane connexons. The well-characterized “scrape loading” technique was used to assess GJIC in HeLa cells expressing wt Cx43. It was found that ACT1 significantly increased GJIC (p<0.05) in HeLa Cx43 cells relative to control and parental, non-connexin expressing cells (FIG. 2).

Functional and biochemical assays were used to assess whether the predicted reduction in free-membrane connexons occurs. First, connexon channel (hemichannel) function was assayed using EtdBr uptake in live cells as described by Saez and co-workers. Consistent with the reduction in hemichannel activity predicted by the model, it was found that ACT1 significantly decreases EtdBr uptake (p<0.05) in contacting HeLa Cx43 cells relative to controls and non-connexin expressing HeLa cells (FIG. 2). In a variant of this experiment, the cells were also cultured at low density such that no GJs could form. Again consistent with the model, in the absence of GJs for free connexons in the membrane to be recruited to, ACT1 had no affect (FIG. 2).

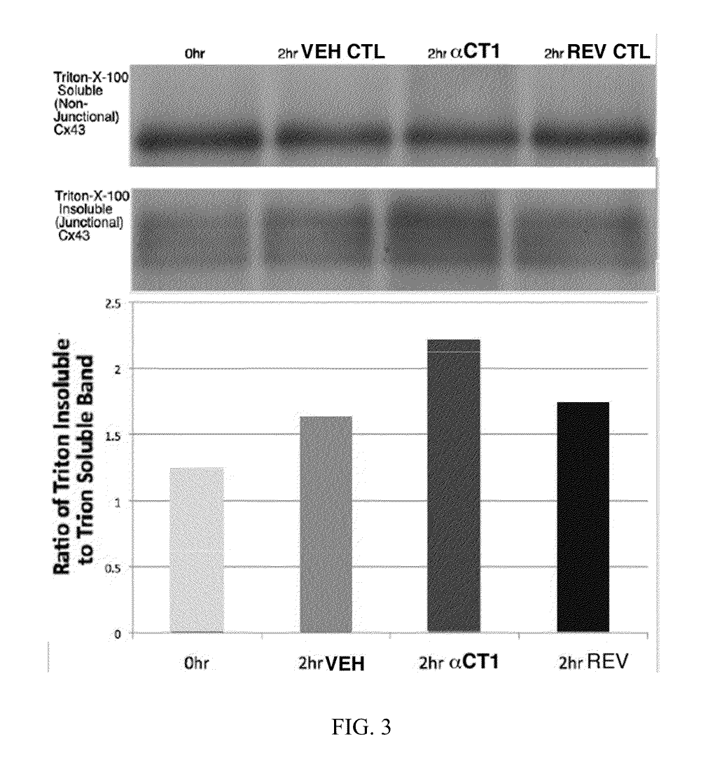

The second approach was to use the surface biotinylation assay of connexon density in the membrane first described by Musil and Goodenough. Here, a pulse of biotin labels proteins exposed at the external surface of the membrane. After allowing 1-4 hrs for biotin-labeled Cx43 to become recruited to GJs, a lysate from the pulsed cells is solubilized by Triton-x-100 detergent into single membrane and GJ fractions, each of which is pulled with streptavidin and then blotted for Cx43. The method provides a convenient quantitative assay of the transition of Cx43 from the membrane into GJs. However, well-controlled data indicating that ACT1 prompts a greater shift of biotin-tagged Cx43 from the detergent-soluble fraction into the detergent-insoluble GJ fraction has recently been obtained (FIG. 3).

b) Differential Effects of Cx43/ZO-1 Interaction on Cx43-Mediated Adhesion:

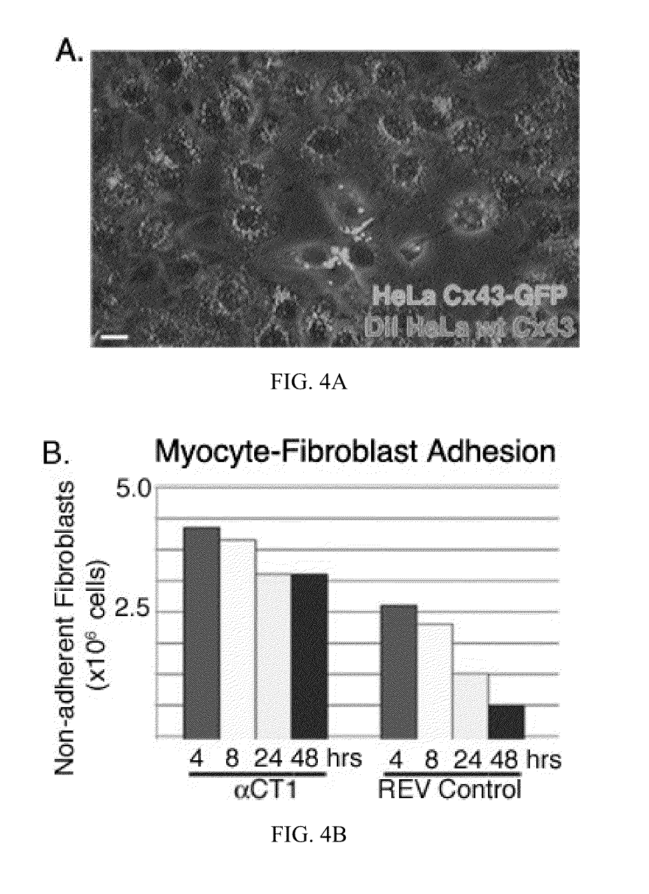

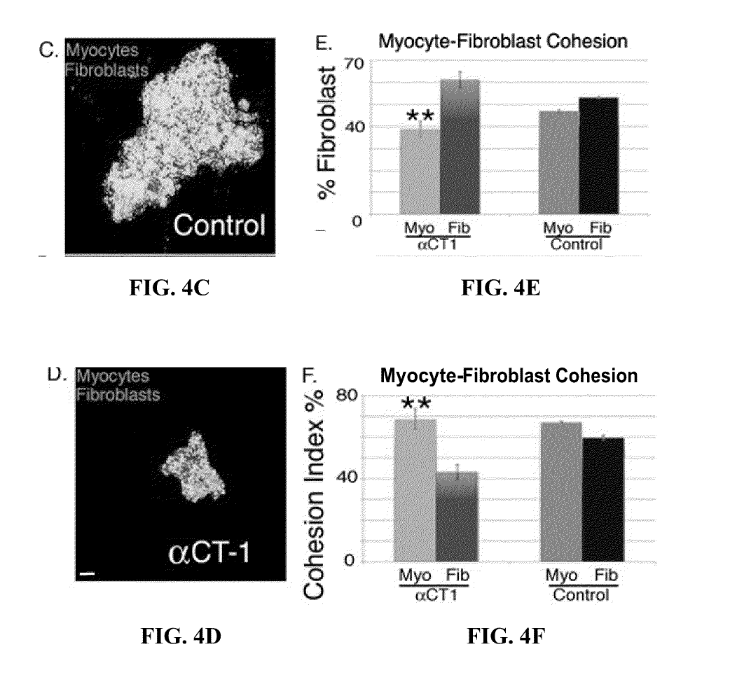

HeLa Cx43-GFP cells (i.e., cells expressing ZO-1 PDZ2 binding incompetent Cx43) do NOT form GJs with HeLa normal Cx43 cells (i.e., cells expressing ZO-1 PDZ2 binding competent Cx43) (FIG. 4A). In another approach neonatal rat ventricular myocytes (NVRMs) were cultured as monolayers and the propensity of added dispersed NCRFs to adhere to this layer was measured. In this assay, ACT1 decreased the adherence of NCRFs to NVRMs (FIG. 4B). In the second approach, green CellTracker-tagged fibroblasts are mixed with orange CellTracker-tagged myocytes and cultured as aggregates (FIG. 4C,D). Consistent with what had been observed in the first assay, data indicated that ACT1 treatments resulted in increased exclusion/loss of NCRFs from the aggregates (FIG. 4E). More interesting yet, relative to control aggregates, myocytes and fibroblasts in aggregates exposed to ACT1 tend to segregate into cohesive domains of like cells (FIG. 4F). Disclosed herein, and supported by the disclosed data, the regulation of connexon aggregation via ZO-1 PDZ2 can dynamically adjust the level of differential adhesivity between cells in heterocellular contacts (e.g., fibroblasts and myocytes).

What is claimed is:

1. A method of treating a subject for membrane excitability comprising administering a PDZ2 targeting modality.

2. The method of claim 1, wherein the PDZ2 targeting modality comprises a conservatively modified variant, amino acid enantiomer or analogue of ACT1 peptide.

3. The method of claim 2, wherein the conservatively modified variant of ACT1 peptide comprises the ACT1 peptide.

4. The method of claim 1, wherein the membrane excitability is of the heart, nervous system, muscle, uterus.

5. The method of claim 4, wherein the subject is being treated for heart attack, epileptic seizure, irritable bowel syndrome, or problematic child birth.

6. The method of claim 1, wherein the PDZ2 targeting modality is delivered orally, intravenously, with an implantable biodegradable matrice, with a gel, with a patch, with a methyl cellulose patch with a wafer, by direct bolus injection into tissues, through a multifunctional polymer, through a micro-/nanoparticulate drug, through a polyion complex, through a liposome, in conjunction with protease inhibitors, a slow release implantable device, catheter-based approaches, through an implantable stent, or through an expanding device.

7. The method of claim 1, wherein the membrane excitability is associated with a tissue arrhythmia.

8. The method of claim 7, wherein the tissue arrhythmia is a cardiac arrhythmia.

9. The method of claim 8, wherein the cardiac arrhythmia is ventricular tachycardia, ventricular fibrillation, atrial fibrillation, bradycardia, tachycardia, automaticity defect, re-entrant arrhythmia, fibrillation, or triggered beats, premature Atrial Contractions, wandering Atrial pacemaker, Multifocal atrial tachycardia, Atrial flutter, Atrial fibrillation, Supraventricular tachycardia, AV nodal reentrant tachycardia is the most common cause of Paroxysmal Supra-ventricular Tachycardia, Junctional rhythm, Junctional tachycardia, Premature junctional complex, Wolff-Parkinson-White syndrome, Lown-Ganong-Levine syndrome, Premature Ventricular Contractions (PVC) sometimes called Ventricular Extra Beats, Accelerated idioventricular rhythm, Monomorphic Ventricular tachycardia, Polymorphic ventricular tachycardia, Ventricular fibrillation, First degree heart block, which manifests as PR prolongation, Second degree heart block, Type 1 Second degree heart block, Type 2 Second degree heart block, or Third degree heart block.

10. The method of claim 1, wherein the membrane excitability is associated with an electrical pathophysiology, wherein electrical pathophysiolgy is a Long QT syndrome, Short QT syndrome, Brugada syndrome, several accessory pathway disorder, Wolff-Parkinson-White syndrome (WPW), Hypertrophic Cardiomyopathy, epilepsy, Autosomal dominant nocturnal frontal lobe epilepsy, Benign centrotemporal lobe epilepsy of childhood, Benign occipital epilepsy of childhood, Catamenial epilepsy, Childhood absence epilepsy, Dravet's syndrome, Frontal lobe epilepsy, Juvenile absence epilepsy, Juvenile myoclonic epilepsy, Lennox-Gastaut syndrome, Primary reading epilepsy, Progressive myoclonic epilepsy, Rasmussen's encephalitis, Symptomatic localization-related epilepsies, Temporal lobe epilepsy, or West syndrome.

11. The method of claim 1, wherein the PDZ2 targeting modality comprises a formulation that delivers 0.001 to 1000 mg per kg body weight to the area of membrane excitability or a reentrant activity.

12. The method of claim 6, wherein the delivery occurs by being placed against the external surface of the heart, in the pericardial sac, the pleural space or through inhalation into the lungs.

13. The method of claim 1, further comprising administering a second arrhythmia treatment.

14. The method of claim 13, wherein the second arrhythmia treatment comprises administering Quinidine, Procainamide, Disopyramide, class Ib drug, Lidocaine, Phenyloin, Mexiletine, class Ic drug, Flecamide, Propafenone, Moricizine, class II drug, Propranolol, Esmolol, Timolol, Metoprolol and Atenolol, class III drug, Amiodarone, Sotalol, Ibutilide and Dofetilide, class IV drug, Verapamil, Diltiazem and class V drug, Adenosine, or Digoxin, performing an Anticoagulant therapy, electrical treatment, electrical cautery, cryo-ablation, radio frequency ablation, implantable cardioverter-defibrillator, or implantable pacemaker.

15. The method of claim 13, wherein the second arrhythmia treatment comprises carbamazepine, clorazepate (Tranxene) clonazepam (Klonopin), ethosuximide (Zarontin), felbamate (Felbatol), fosphenyloin (Cerebyx), gabapentin (Neurontin), lamotrigine (Lamictal), levetiracetam (Keppra), oxcarbazepine (Trileptal), phenobarbital (Luminal), phenyloin (Dilantin), pregabalin (Lyrica), primidone (Mysoline), tiagabine (Gabitril), topiramate (Topamax), valproate semisodium (Depakote), valproic acid (Depakene), zonisamide (Zonegran), clobazam (Frisium) and vigabatrin (Sabril), retigabine, brivaracetam, and seletracetam, diazepam (Valium, Diastat) and lorazepam (Ativan), Paral, midazolam (Versed), and pentobarbital (Nembutal), acetazolamide (Diamox), progesterone, adrenocorticotropic hormone (ACTH, Acthar), various corticotropic steroid hormones (prednisone), bromide, ketogenic diet, electrical stimulation, vagus nerve stimulation, responsive neurostimulator system (rns), deep brain stimulation, invasive or noninvasive surgery, avoidance therapy, warning systems, alternative or complementary medicine.

16. A formulation of a PDZ2 targeting modality comprising a patch for directly delivery to a heart.

17. A device comprising a long term release mechanism for delivery of a PDZ2 targeting modality.

18. A method of producing the PDZ2 targeting modality of claim 16.
